(19) United States
(12) Patent Application Publication
(10) Pub. No.: US 2004/0209348 A1
(43) Pub. Date: Oct. 21, 2004

(54) Polymerase chain reaction apparatus

(76) Inventor: Ulf Lanz, Erlangen (DE)

Correspondence Address: Brinks Hofer Gilson & Lione, P.O. Box 10395, Chicago, IL, 60610, US

(21) Appl. No.: 10/810,265

(22) Filed: Mar. 24, 2004

(30) Foreign Application Priority Data
Mar. 27, 2003 (DE) 10313898.6

(51) Int. Cl. C12M 1/38

(52) U.S. Cl. 435/286.5; 435/287.2; 435/303.1

(57) ABSTRACT

A PCR apparatus has a specimen chamber, a heating conduit which communicates with the specimen chamber, a cooling conduit which communicates with the specimen chamber, and a pumping device for pumping a gaseous or liquid medium through the heating conduit and/or the cooling conduit to the specimen chamber. The PCR apparatus further has a heating device, disposed separately from the cooling conduit, which communicates with the heating conduit and heats the medium located in the heating conduit c. The PCR apparatus has further a mixing device, which communicates with the heating conduit and with the cooling conduit, such that a ratio between a volume of the medium flowing per unit of time through the heating conduit and a volume of the medium flowing per unit of time through the cooling conduit to the specimen chamber is adjustable.

### BACKGROUND

The invention relates to a Polymerase Chain Reaction (PCR) apparatus by which a PCR specimen can be subjected to a temperature cycle.

PCR apparatuses are typically used to replicate gene sequences by molecular-biological copying of a particular gene. This replication can serve, among other purposes, to enhance a detectability of these gene sequences during a detection process. The enhanced detectability for instance makes it possible, even with specimens of small volume or of initial low genetic concentration, to perform genetic analysis, which can then be employed for medical diagnosis of a corresponding organism from which the specimen was taken.

The PCR process is based on generating identical copies of individual DNA strands of the gene sequences present in the specimen. As such, the specimen is typically subjected to a thermal cycle, which first causes cleavage of the two-strand DNA sequences via an increased temperature and then, via a reduced temperature, restores the two-strand DNA structure on the basis of the individual strands previously cleaved off and using DNA building blocks that are added to the specimen. In the restoration, using the DNA building blocks, identical copies of the individual DNA strands are put together, which is similar to or the same as copying the DNA sequences. By repeating the thermal cycle multiple times, an exponential replication of the DNA sequences contained in the specimen is achieved.

For the polymerase chain reaction (PCR), as described above, to be successful in replicating DNA sequences, it is essential to maintain one predetermined thermal cycle per DNA sequence and per specimen composition. This thermal cycle is accomplished by means of a so-called PCR apparatus, also known as a “Thermal Cycler” or “Thermocycler”. The specimens are typically kept in special reagent vessels, such as so-called “Eppendorf” vessels, which on one hand make it possible to handle the specimens while maintaining the requisite purity and on the other hand may have suitable thermal properties for achieving the thermal cycle. The PCR apparatus has a specimen chamber, suitable for holding such vessels, which is heated or cooled by heating or cooling elements, i.e. conduits, ducts or channels, respectively, and is monitored by a temperature sensor.

From U.S. Pat. No. 5,455,175, a PCR apparatus is known in which ambient air is used as the heating and cooling medium. Heating the PCR specimen is done either by heating the air that flows through the specimen chamber or by irradiating the specimen with a heat lamp. For cooling, the PCR apparatus allows the air in the specimen chamber and on the heating lamp or heating coil to escape into the environment and be replaced with cooler ambient air. The escape of the heated air causes the surroundings of the PCR apparatus, such as a laboratory, to heat up, which is typically unwanted.

From U.S. Pat. No. 6,482,615, a PCR apparatus is known in which once again ambient air is used as the cooling or heating medium; the ambient air is blown in a high-velocity air stream through the specimen chamber. For heating, the ambient air is heated by a heating element in an intake region of the apparatus. For cooling, the heating element is deactivated, and the heated air located in the specimen chamber and around the heating element is blown out into the surroundings and in the process replaced with cool ambient air. The high velocity of the air stream serves to effect especially efficient heat exchange. Because of the escape of the heated air, the surroundings of the PCR apparatus are heated, which is typically unwanted. Moreover, the high-velocity air stream produces noise, which again is not typically wanted.

### OBJECT AND SUMMARY

The present invention is defined by the following claims. This description summarizes some aspects of the present embodiments and should not be used to limit the claims.

An object is to disclose a PCR apparatus which, being intended for use in laboratory surroundings, produces the least possible amount of heat and noise. A further object is to disclose such a PCR apparatus which is as simple as possible in its construction.

One concept is to disclose a PCR apparatus, with a specimen chamber, a heating conduit which communicates with the specimen chamber, a cooling conduit which communicates with the specimen chamber, a pumping device for pumping a gaseous or liquid medium through the heating conduit and/or the cooling conduit to the specimen chamber, and a heating device which communicates with the heating conduit, which is embodied such that the medium located in the heating conduit is heatable by the heating device, and in which the cooling conduit is disposed separately from the heating device.

One characteristic for cooling the specimen chamber is delivering a medium to be cooled and which may not come into contact with the heating device. One consequential advantage is that the heating device may not need to be cooled jointly with the specimen chamber in order to attain a lower temperature of the thermal cycle. Instead, the heating device can be left in an environment of the heated medium, while the cooling can be done completely separately from the heating device and the medium surrounding the heating device. This may simultaneously lessen the heating of the environment of the PCR apparatus, since the heat of the heating device does not have to be dissipated during the cooling. Furthermore, the cooling medium, being supplied separately from the heating device, is more rapidly available and hence may enable a substantially faster cooling of the specimen chamber. Further, a time thus saved can be utilized to reduce the flow velocity of the cooling medium and thereby to reduce the noise emitted as a result of the flow.

In an advantageous feature, the PCR apparatus may have a mixing device which communicates with the heating conduit and with the cooling conduit and may be embodied in such a way that a ratio between a volume of the medium flowing per unit of time through the heating conduit and a volume of the medium flowing per unit of time through the cooling conduit to the specimen chamber can be varied or adjusted. A use of such mixing device has an advantage that by varying a mixture ratio in question, a desired temperature level can be established in the specimen chamber without having to know the temperature of the medium in the cooling conduit or the heating conduit or the heating device having to have a defined temperature. Moreover, time constants in terms of heating or cooling the heating device may be eliminated, because they can be compensated for at any time by means of the mixture ratio. Compensating for timing effects of the heating device is especially advantageous when a lower heated temperature level is reached from a higher heated temperature level.

Still further, the mixing device may have an especially uncomplicated construction. The mixing device may be a single apparatus component, instead of a plurality of apparatus components, such as valves, which may be sufficient for metering the medium. The mixing device may be advantageous from a standpoint of regulation as well. Specifically, for regulating the temperature, regulating or controlling only the mixing device as the sole apparatus component may be sufficient. The mixing device may prove advantageous in regulating the heat output of the heating device in addition, which can be done in an uncomplicated way in a form of purely electrical regulation.

In a further advantageous feature, the PCR apparatus may be embodied such that the ambient air can be used as the medium. As such, no special provisions may be needed for sealing off the PCR apparatus to prevent the medium from being lost as a result of leaks. Moreover, the ambient air as the medium is readily available at all times, without limitation, and need not be separately procured and introduced.

In another advantageous feature, the mixing device may include a valve, by which the volume of the medium flowing through the cooling conduit or the volume of the medium flowing through the heating conduit per unit of time can be varied. This additional valve may make it possible to provide and realize the mixing device in an especially uncomplicated way.

In a further advantageous feature, the PCR apparatus has a temperature sensor, which may be embodied in such a way that a temperature in the specimen chamber can be measured in a contact-less fashion. An infrared detector, for instance, can be used for this purpose. As such, a temperature of the specimen vessels, which are directly in contact with the PCR specimen, may be measured. As a result, an actual level of the specimen temperature can be measured in a substantially direct way. At the same time, the contact-less measurement may offer a substantial security against measurement errors originating from contamination or from dissimilar specimen vessel materials and furthermore makes constant measurement conditions possible, regardless of changes in the specimen vessels from one PCR cycle to another.

Further advantages will become apparent from the dependent claims and the description of exemplary embodiments.

Exemplary embodiments of the invention will be described below in conjunction with the figures.

### BRIEF DESCRIPTION OF THE DRAWINGS

FIG. 1 illustrates an embodiment of a PCR apparatus with a separate heating conduit and cooling conduit; and

FIG. 2 illustrates another embodiment of a PCR apparatus with a mixing device arranged differently than that of FIG. 1.

### DESCRIPTION OF THE PREFERRED EMBODIMENTS

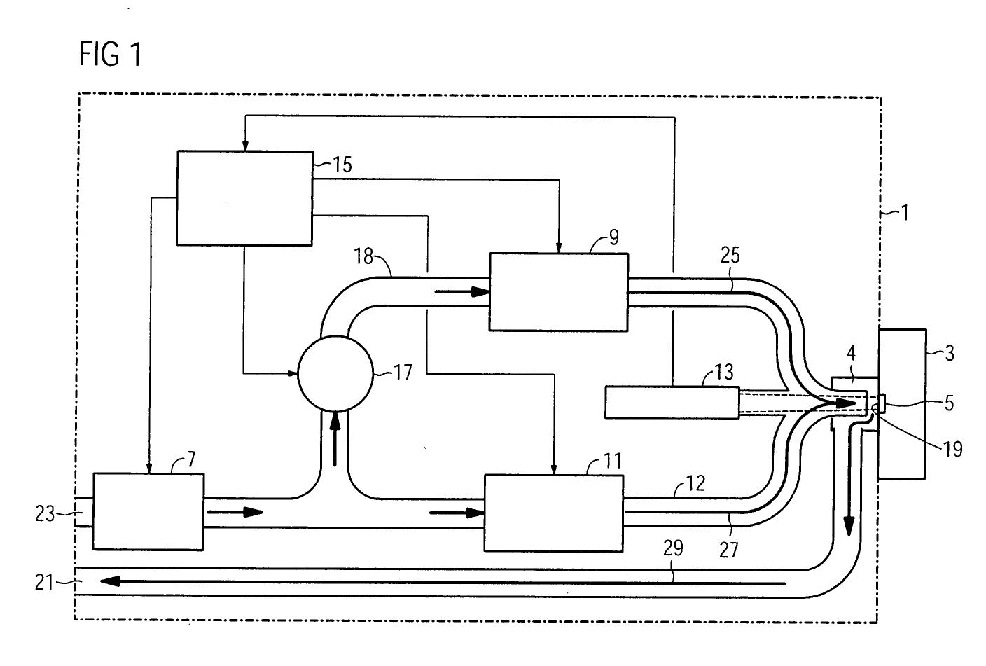

In FIG. 1, an embodiment of a PCR apparatus with a separate heating conduit and cooling conduit is shown. The PCR apparatus 1 is used to generate a thermal cycle to which a specimen 5, located in a specimen cassette 3, cartridge or magazine, is to be subjected. The specimen 5 contains a genetic specimen taken from a living organism, along with all DNA building blocks and other microbiological substances that are used for the PCR process, which together are known as a PCR kit.

In an advantageous feature, the specimen cassette 3 has a sealed specimen volume for the specimen 5, and this volume at first contains only the genetic specimen, without the PCR kit. Typical substances making up the PCR kit are also located in the specimen cassette 3, but separately from the specimen volume. Inside the specimen cassette 3, the substances are mixed with or added to the specimen 5, either at the beginning or during the PCR process, via a suitable mechanism or hydraulics. For actuating the hydraulics of the specimen cassette 3, the cassette 3 has couplings on the outside that can be engaged with the PCR apparatus 1. These couplings may for instance be Bowden cables, gear wheels, or dies that can be pressed into them. A specimen cassette 3 of this kind can contain the entire PCR kit in a form of a disposable product, and the specimen 5 is then added by a person, such as a laboratory technician or a medical-technical assistant (MTA).

The PCR apparatus 1 has a specimen chamber 4, into which a specimen 5 in a suitable specimen vessel can be inserted, or with which a specimen cassette 3 can be made to communicate. The PCR apparatus 1 may bring about a pre-determinable thermal cycle in the specimen chamber 4. The specimen cassette 3 can be embodied such that the specimen 5 located in the cassette 3 lies directly under the surface 19 of the cassette 3 that is made to communicate with the specimen chamber 4. The specimen cassette 3 can also be embodied in such a way that a medium from the specimen chamber 4 can flow through suitable conduits in the cassette 3.

The PCR apparatus 1 has a pumping device 7 by which a gaseous or liquid medium can be pumped through the specimen chamber 4 via a conduit system. In an advantageous embodiment, ambient air that is aspirated through an inflow opening 23 by the pumping device 7 is used as the medium. The conduit system has a further outflow opening 21, through which the ambient air can leave the conduit system again. To avoid excessive heating of the surroundings of the PCR apparatus 1, the out-flowing air can be returned entirely or partially to the conduit system again. With a view to efficient cooling of the specimen chamber 4, however, consideration must be given to the fact cool ambient air may need to flow in the conduit system. Without active cooling, a completely closed conduit system may not be feasible.

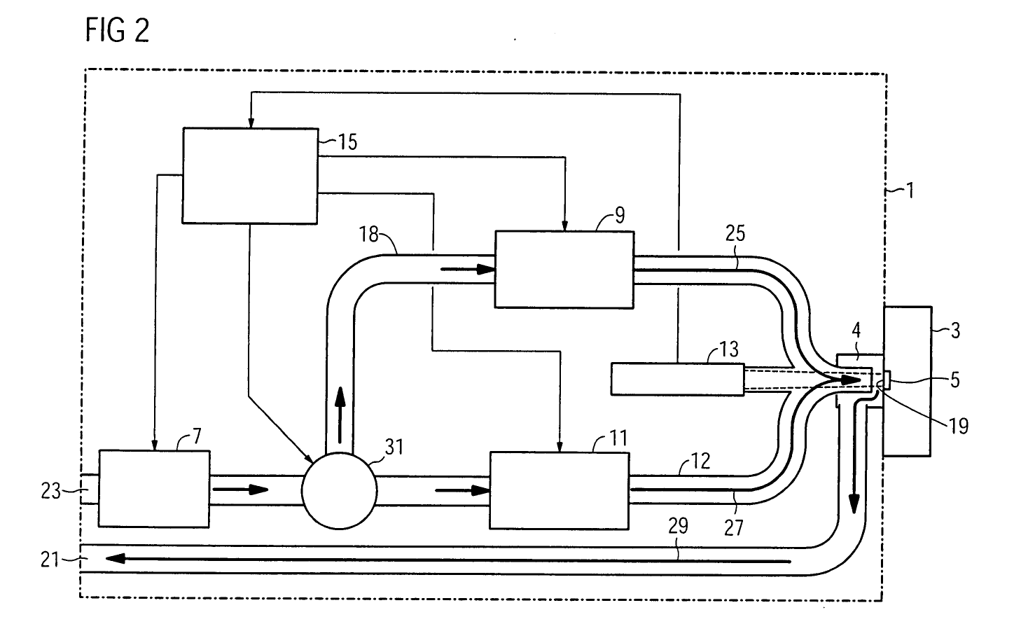

The medium is pumped by the pumping device 7 through a cooling conduit 18 to the specimen chamber 4, as indicated by arrows in FIGS. 1 and 2. In an advantageous embodiment, a cooling device 9 communicating with the cooling conduit 18 may be embodied to actively cool the medium flowing through the cooling conduit 18. For that purpose, the cooling device 9 can have a heat exchanger, which extracts heat from the medium using a coolant. The cooled medium may flow in a form of a cooled stream 25 to the specimen chamber 4.

The medium is also pumped to the specimen chamber 4 by the pumping device 7 through a heating conduit 12, which communicates with a heating device 11. The heating device 11 may be embodied to heat the medium flowing through the heating conduit 12. For that purpose, the heating device 11 can for instance have a heat exchanger, a heat lamp, or a heating coil. The heated medium may flow in a form of a heating stream 27 to the specimen chamber 4.

In the specimen chamber 4, the heating stream 27 and the cooling stream 25 may mix, resulting in a temperature that may be dependent both on a current temperature at the specimen chamber 4 and on the mixture ratio of the two streams. The medium may leave the specimen chamber 4 in a mixed stream 29, which can escape from the conduit system through the outflow opening 21.

A temperature of the specimen 5, or the surface 19 of the specimen cassette 3, may be measured by a temperature sensor 13. As a result, the temperature measurement may be independent of potential contaminations of the surface 19 and may also not be dependent on an exact positioning of the specimen vessel or specimen cassette 3. Furthermore, the temperature measured at the surface 19 is maximally independent of the temperature of the medium flowing through the specimen chamber 4. As a result, inaccuracies in measuring the specimen temperature that are due to the thermal capacity of the specimen cassette 3 may be minimized. In the embodiment shown, the temperature sensor 13 is embodied as an infrared detector, by which the temperature of the surface 19 can be measured in a contact-less fashion. The infrared detector may detect the infrared specimen temperature at a viewing angle that is predetermined by the optics of the detector. The viewing angle is represented by dashed lines in FIG. 1.

In order to establish a pre-determinable temperature in the specimen chamber 4, an output signal of the temperature sensor 13 is delivered to a temperature regulating device 15. The temperature regulating device or temperature regulator 15 is thus informed of the current temperature in the specimen chamber 4, and is additionally supplied with the desired temperature cycle by means of a memory or signal input, not shown. Thus, the temperature regulating device 15 has the requisite information to enable regulating the temperature as a controlled variable in accordance with the predetermined temperature cycle.

As an additional regulating parameter, the heating output may be varied via a signal connection with the heating device 11. For that purpose, it may suffice in principle to control the heating output. In an advantageous embodiment, however, the heating output can also be regulated. As such, a signal connection between the temperature regulating device 15 and the heating device 11 may supply a signal to the temperature regulating device 15 that may include information about the current temperature of the heating device 11. As a further regulating parameter, the temperature regulating device 15 can control or regulate an optionally provided cooling device 9, analogously to the heating device 11.

One characteristic of the PCR apparatus 1 is that the cooling stream 25 flows separately from the heating device 11. As a result, the temperature of the cooling stream 25 supplied to the specimen chamber 4 is not affected by the current temperature of the heating device 11. In particular, in the cooling conduit 18, the medium which is not in any way heated by the heating device 11 is available directly and without delay for delivery to the specimen chamber 4.

To enable regulating the temperature suitably flexibly and quickly, the temperature regulating device 15 may make the mixture ratio of the cooling stream 25 and the heating stream 27 available as a definitive regulated parameter. More precisely, via a mixing device, the temperature regulating device 15 regulates the ratio between a volumetric flow, or in other words a volume flowing per unit of time, of the medium, whether cool or heated, that is flowing to the specimen chamber 4. In the embodiment shown, the mixing device is embodied as a valve which varies the volumetric flow of the cooling stream 25. In a further embodiment, not shown, the valve 17 may instead vary the volumetric flow of the heating stream 27. If the volumetric flow of the cooling stream 25, for instance, is varied by the valve 17, then, as a result, the cooling conduit 18 can be considered as a bypass around the heating conduit 12, and the valve 17 can be called a bypass valve.

If the valve 17 is disposed as a bypass valve as described, then the mixture ratio of the two streams in the specimen chamber 4 may affect the valve position on one hand and a hydro-mechanical construction (design) or layout of the rest of the conduit system on the other hand. The hydro-mechanical embodiment of a branch between the cooling conduit 18 and the heating conduit 12, for instance, offers an opportunity of allowing a predominant volumetric flow of medium to be in a direction of the valve 17. As a result, when the valve 17 is open, the medium flows predominantly through the conduit in which the valve 17 is disposed. However, if the valve 17 is closed, this medium flows through the conduit in which the valve 17 is not disposed, counter to the hydro-mechanical embodiment of the branch. This kind of hydro-mechanical embodiment of the conduit system may enable the widest possible range of variation for the mixture ratio as a regulating parameter.

A further hydro-mechanical variable may reside in an internal flow resistance of the heating device 11 or, optionally, of the cooling device 9. In a further advantageous embodiment, the PCR apparatus 1 may have a heating device 11 with greater hydro-mechanical resistance than the cooling conduit 18. As a result, in principle, the volumetric flow of the cooling stream 25 may predominate over that of the heating stream 27. The valve 17 in this embodiment is disposed in the cooling conduit 18, so that when the valve 17 is closed, the medium may flow exclusively through the heating conduit 12, while with the valve 17 open, the medium may flow predominantly through the cooling conduit 18.

As a further regulating parameter, the temperature regulating device 15 may control or regulate the pumping device 7, in order to vary the cooling or heating output in the specimen chamber 4. The cooling or heating output may be varied by varying a pumping output, which is associated with the volumetric flow of the medium to the specimen chamber 4. Increasing the pumping output involves an increase in the output of the heating device 11, in order to bring about a more-effective heating of the specimen chamber 4.

In the embodiment shown, the ambient air may be used as the medium. The ambient air can be aspirated through the inflow opening 23 and blown out through the outflow opening 21. In particular, using ambient air as the medium makes it possible to use a conduit system that is not closed.

As a result of the mixed stream 29 escaping through the outflow opening 21, the environment of the PCR apparatus 1, such as a laboratory, is heated. However, this heating is minimized by providing that the heating device 11 is disposed separately from the cooling conduit 18 or cooling stream 25, so that during cooling phases in the predetermined temperature cycle, the air located in the heating conduit 12 or in the heating device 11 is not blown out. Instead, the heated air can remain in the heating conduit 12 and be used for the next temperature increase in the temperature cycle. As a result, heating of the environment of the PCR apparatus 1 from unnecessary cooling down of the heating device 11 and the heating conduit 12 may be minimized.

Furthermore, cooling air for cooling down the specimen chamber 4 is immediately available, rather than only after the heating device 11 has cooled down. It is therefore possible, despite the requisite dissipation of the heat of the heating device 11, to dispense with an increased pumping output of the pumping device 7 for faster cooling, which means less noise is emitted by the flowing air.

Still further embodiments of the invention can be attained for instance by modifying the disposition of the pumping device 7. For instance, the pump may also be disposed on the side of the out-flowing mixed stream 29. For the mode of operation of the conduit system, in principle, there may not be a difference whether the pumping device 7 pumps the medium to the specimen chamber 4 or aspirates it away from it.

In FIG. 2, a further embodiment is shown, which except for the disposition or location of the mixing device has the same characteristics and uses the same reference numerals. However, in contrast to the embodiment described above in conjunction with FIG. 1, the mixing device is embodied not as a valve but rather as a mixing valve 31.

The mixing valve 31 has the function of being capable of varying the volumetric flow of medium flowing through the heating conduit 12 and the volumetric flow through the cooling conduit 18 simultaneously but in opposite directions; that is, if one volumetric flow is increased by triggering the mixing valve 31, the other volumetric flow is simultaneously decreased. In this respect, the mode of operation of the mixing valve 31 is like that of a conventional one-armed mixing battery of the kind known in a plumbing field. This mixing valve 31 may enable substantially rapid variations in the mixing ratio of the cooling stream 25 and heating stream 27 over a maximum possible range of variation, namely from a volumetric flow solely through the heating conduit 12 to a volumetric flow solely through the cooling conduit 18, regardless of the hydro-mechanical conditions in the rest of the conduit system or in the heating device 11 or the cooling device 9.

What is claimed is:

1. A PCR apparatus comprising:
a specimen chamber;
a heating conduit which communicates with the specimen chamber;
a cooling conduit which communicates with the specimen chamber;
a pumping device for pumping a gaseous or liquid medium through the heating conduit and the cooling conduit to the specimen chamber;
a heating device, disposed separately from the cooling conduit, which communicates with the heating conduit and is operable to heat the medium located in the heating conduit; and
a mixing device which communicates with the heating conduit and with the cooling conduit such that a ratio between a volume of the medium flowing per unit of time through the heating conduit and a volume of the medium flowing per unit of time through the cooling conduit to the specimen chamber is adjustable by the mixing device.

2. The PCR apparatus as in claim 1., wherein the medium is ambient air.

3. The PCR apparatus of claim 2, wherein the cooling conduit is opened such that the ambient air flows in.

4. The PCR apparatus as in claim 1, further comprising a temperature sensor to measure a temperature in the specimen chamber.

5. The PCR apparatus as in claim 4, wherein the temperature sensor is an infrared detector.

6. A PCR apparatus comprising:
a specimen chamber;
a heating conduit which communicates with the specimen chamber;
a cooling conduit which communicates with the specimen chamber;
a pumping device for pumping a gaseous or liquid medium through the heating conduit and the cooling conduit to the specimen chamber;
a heating device, disposed separately from the cooling conduit, which communicates with the heating conduit and is operable to heat the medium located in the heating conduit; and
a mixing device, disposed in one of the cooling conduit and the heating conduit, such that a ratio between a volume of the medium flowing per unit of time through the heating conduit and a volume of the medium flowing per unit of time through the cooling conduit to the specimen chamber is adjustable by the mixing device.

7. The PCR apparatus as in claim 6, wherein the medium is ambient air.

8. The PCR apparatus of claim 7, wherein the cooling conduit is opened such that the ambient air flows in.

9. The PCR apparatus as in claim 6, further comprising a temperature sensor to measure a temperature in the specimen chamber.

10. The PCR apparatus as in claim 9, wherein the temperature sensor is an infrared detector.

11. A PCR apparatus comprising:
a specimen chamber;
a heating conduit which communicates with the specimen chamber;
a cooling conduit which communicates with the specimen chamber;
a pumping device for pumping a gaseous or liquid medium through the heating conduit and or the cooling conduit to the specimen chamber;
a heating device, disposed separately from the cooling conduit, which communicates with the heating conduit and is operable to heat the medium located in the heating conduit; and
a mixing device which communicates with the heating conduit and with the cooling conduit, such that a ratio between a volume of the medium flowing per unit of time through the heating conduit and a volume of the medium flowing per unit of time through the cooling conduit to the specimen chamber is adjustable by the mixing device,
wherein the mixing device includes a valve, which varies one of the volume of the medium flowing through the cooling conduit and the volume of the medium flowing through the heating conduit.

12. The PCR apparatus as in claim 11, wherein the medium is ambient air.

13. The PCR apparatus as in claim 12, wherein the cooling conduit is opened such that the ambient air flows in.

14. The PCR apparatus as in claim 11, further comprising a temperature sensor to measure a temperature in the specimen chamber.

15. The PCR apparatus as in claim 14, wherein the temperature sensor is an infrared detector.
